(19) United States
(12) Patent Application Publication
(10) Pub. No.: US 2020/0188237 A1
(43) Pub. Date: Jun. 18, 2020

(54) ENDODONTIC FORMULATIONS CONTAINING CYSTEAMINE AND CYSTEAMINE DERIVATIVES AND USES THEREOF

(71) Applicant: University of Florida Research Foundation, Inc., Gainesville, FL (US)

(72) Inventor: Hope L. Feldman, Palm Coast, FL (US)

(21) Appl. No.: 16/630,895

(22) PCT Filed: Jul. 14, 2018
(86) PCT No.: PCT/US2018/042190

§ 371 Date: Jan. 14, 2020

Related U.S. Application Data
(60) Provisional application No. 62/670,527, filed on May 11, 2018.
(60) Provisional application No. 62/532,580, filed on Jul. 14, 2017.

(51) Int. Cl. A61K 6/52; A61K 6/69

(52) U.S. Cl. CPC A61K 6/52; A61K 6/69

(57) ABSTRACT

Endodontic formulations and methods of use are provided. The formulations can be irrigant formulations for irrigating a root canal using an effective amount of Cysteamine or a Cysteamine derivative. The effective amount can be effective to inhibit the proliferation of at least one bacterial infection in the root canal patient. The effective amount can be effective to produce a percent reduction in Enterococcus faecalis in the canal wall of the patient of about 60% or more when administered to the patient. The effective amount can be effective to produce a percent reduction in Fusobacterium nucleatum in the canal wall of the patient of about 65% or more when administered to the patient. The formulations can be decalcifying formulations for reducing a calcification of a tissue in a root canal of a patient in need thereof. The decalcifying ability can be greater than a corresponding formulation such as EDTA and/or NaOCl.

### CROSS-REFERENCE TO RELATED APPLICATIONS

This application claims priority to, and the benefit of, co-pending U.S. provisional application entitled “Endodontic Formulations Containing Cysteamine AND Cysteamine Derivatives and Uses Thereof” having Ser. No. 62/532,580, filed Jul. 14, 2017 and co-pending U.S. provisional application entitled “Endodontic Formulations Containing Cysteamine AND Cysteamine Derivatives and Uses Thereof” having Ser. No. 62/670,527, filed May 11, 2018, the contents of both of which are incorporated by reference in their entirety.

### TECHNICAL FIELD

The present disclosure generally relates to endodontics.

### BACKGROUND

The success of root canal treatment can depend on thorough disinfection of the root canal. A variety of methods have been developed for irrigation, including irrigant formulations such as sodium hypochlorite (NaOCl). Although NaOCl is a widely used irrigant, it has proven to be ineffective in many instances and at currently used dosages. Many companies have discontinued previous concentrations of 5.25% and 6.15% and the current full strength concentration is 8.25%. While 8.25% NaOCl promotes significantly more Enterococcus faecalis reduction than 6.15, there still remains a need for improved irrigant formulations.

The pulp in the dental canal is prone to dystrophic mineralization with calcium; this calcification can be so extensive that the entire root canal system is obliterated. As a result, root canal treatment can become difficult, and in some cases impossible. Calcified root canals are often difficult to locate and treat. Calcium deposits in the root canal can result from prolonged infection, trauma, or just with age. In some instances, the pulp chamber can become completely occluded by calcium deposits. A number of decalcifying treatments are available, including treating the calcified tissue with EDTA or NaOCl. However, there remains a need for improved formulations and methods for decalcifying calcified tissue in the root canal.

### SUMMARY

In various aspects, endodontic formulations containing Cysteamine or a derivative thereof are provided. Methods of using Cysteamine or a derivative thereof in endodontic applications are also provided. Applicants have found that Cysteamine and Cysteamine derivatives can be effective in preventing or inhibiting bacterial proliferation in a root canal and/or can be effective at decalcifying calcified tissues in a root canal. The formulations can therefore be irrigant formulations, decalcifying formulations, or a combination thereof. The formulations can also include one or more additional agents, although in some aspects the formulations can consist essentially of Cysteamine or a derivative thereof.

In various aspects, irrigant formulations and methods of irrigating a root canal wall of a patient in need thereof are provided that overcome one or more of the aforementioned deficiencies. The irrigant formulations include an effective amount of Cysteamine or a derivative thereof. In some aspects, the Cysteamine or Cysteamine derivative is present in an amount from about 8 mg/ml to about 12 mg/ml. Methods of irrigating a root canal wall of a patient are also provided. The methods can include administering an effective amount of a formulation described herein to the patient and/or administering an effective amount of a Cysteamine or Cysteamine derivative to the patient.

In various aspects, decalcifying formulations and methods are provided for reducing the calcification of a tissue in a root canal of a patient in need thereof. In some aspects, the effective amount is effective to reduce a calcification of the tissue when exposed to the formulation for a period of about 48 hours to about 240 hours.

In some aspects, the formulations and methods include a Cysteamine derivative having a structure according to the following formula:

H₂N-A-SH

where A is a substituted or unsubstituted C₁-C₇ alkyl, substituted or unsubstituted C₂-C₇ alkenyl, substituted or unsubstituted C₂-C₇ alkynyl, substituted or unsubstituted C₃-C₇ cycloalkyl, substituted or unsubstituted C₃-C₇ cycloalkenyl, or substituted or unsubstituted C₃-C₇ alkynyl. In some aspects, A is a substituted or unsubstituted C₂-C₅ alkyl.

The formulations contain an effective amount of the Cysteamine or Cysteamine derivative. The methods include administering an effective amount of the Cysteamine, Cysteamine derivative, or a formulation thereof to the patient. In various aspects, the effective amount is effective to reduce the bacterial proliferation in the root canal wall of the patient as compared to a bacterial proliferation of the otherwise same root canal wall when treated with the otherwise same irrigant formulation except without the Cysteamine or derivative thereof. In some aspects, the effective amount is effective to reduce the bacterial proliferation in the root canal wall of the patient as compared to a bacterial proliferation of the otherwise same root canal wall when treated with the otherwise same irrigant formulation except with the Cysteamine or Cysteamine derivative replaced with NaOCl in an amount ranging from about 6.15% to about 8.25% (v/v) based upon the volume of the formulation. The bacterial proliferation can include, for example, Enterococcus faecalis, Fusobacterium nucleatum, and a combination thereof. In some aspects, the effective amount is effective to produce a percent reduction in Enterococcus faecalis in the canal wall of the patient of about 60% or more when administered to the patient. In some aspects, the effective amount is effective to produce a percent reduction in Fusobacterium nucleatum in the canal wall of the patient of about 65% or more when administered to the patient.

The formulations can include a suitable carrier. In some aspects, the formulations can include additional agents such as decalcifying agents, surfactants, preservatives, emulsifiers, penetration enhancers, and other suitable additives commonly used in endodontic formulations. Decalcifying agents can include ethylene diamine tetra acetic acid (EDTA), citric acid, or hydroxy ethylidene-bisphosphonate (HEBP).

Other systems, methods, features, and advantages of irrigant formulations and methods of use thereof will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Further aspects of the present disclosure will be readily appreciated upon review of the detailed description of its various aspects, described below, when taken in conjunction with the accompanying drawings.

FIG. 1 is a schematic representation of the experimental procedure for Example 1.

FIG. 2 is a bar graph of the percent reduction in E. faecalis as a function of the irrigant treatment.

FIG. 3 is a bar graph of the percent reduction in F. nucleatum as a function of the irrigant treatment.

### DETAILED DESCRIPTION

In various aspects, irrigant formulations and methods of irrigating a root canal are provided. The formulations include an effective amount of Cysteamine or a Cysteamine derivative. The Cysteamine or Cysteamine derivative can provide for inhibition of bacterial growth in the root canal wall, leading to better endodontic outcomes.

Before the present disclosure is described in greater detail, it is to be understood that this disclosure is not limited to particular aspects described, and as such may, of course, vary. It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant specification should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

Although any methods and materials similar or equivalent to those described herein can also be used in the practice or testing of the present disclosure, the preferred methods and materials are now described. Functions or constructions well-known in the art may not be described in detail for brevity and/or clarity. Aspects of the present disclosure will employ, unless otherwise indicated, techniques of endodontics, chemistry, material science and the like, which are within the skill of the art. Such techniques are explained fully in the literature.

It should be noted that ratios, concentrations, amounts, and other numerical data can be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In some aspects, the term “about” can include traditional rounding according to significant figures of the numerical value. In addition, the phrase “about ‘x’ to ‘y’”, where ‘x’ and ‘y’ are numerical values, includes “about ‘x’ to about ‘y’”.

Definitions

Unless defined otherwise, all technical and scientific terms used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

The articles “a” and “an,” as used herein, mean one or more when applied to any feature in aspects of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used.

The term “smear layer,” as used herein, refers to the complex accumulation of organic and inorganic debris resulting from the mechanical preparation of a tooth surface. The smear layer includes cutting debris, tooth particles, microorganisms, necrotic material, and other substances resulting from preparation, and can include a superficial layer on the surface of a prepared tooth along with a layer or layers that are packed into the adjacent dentinal tubules at varying depths.

The term “disinfectant”, as used herein, refers collectively to compositions that are able to suppress or eliminate bacterial or other microorganisms found in endodontic or periodontic sites. The term “disinfectant” includes antibiotics as that term is understood in pharmaceutical science.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups.

In some embodiments, a straight chain or branched chain alkyl has 30 or fewer carbon atoms in its backbone (e.g., C₁-C₃₀ for straight chains, C₃-C₃₀ for branched chains), 20 or fewer, 12 or fewer, or 7 or fewer. Likewise, in some embodiments cycloalkyls have from 3-10 carbon atoms in their ring structure, e.g. have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a hosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, or from one to six carbon atoms in its backbone structure. Likewise, “lower alkenyl” and “lower alkynyl” have similar chain lengths. Throughout the application, preferred alkyl groups are lower alkyls. In some embodiments, a substituent designated herein as alkyl is a lower alkyl.

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

The terms “alkenyl” and “alkynyl”, refer to unsaturated aliphatic groups analogous in length and possible substitution to the alkyls described above, but that contain at least one double or triple bond respectively.

Endodontic Formulations

The formulations described herein contain an effective amount of Cysteamine or a Cysteamine derivative in a pharmaceutical carrier appropriate for administration to an individual in need thereof. The formulations can include irrigant formulations and/or decalcifying formulations. The formulations can be administered topically to the root canal wall of the patient in need thereof.

A variety of irrigant formulations are provided for reducing a bacterial proliferation in a root canal wall of a patient in need thereof. The irrigant formulation can include an effective amount of Cysteamine or a derivative thereof to reduce the bacterial proliferation in the root canal wall of the patient as compared to a bacterial proliferation of the otherwise same root canal wall when treated with the otherwise same irrigant formulation except without the Cysteamine or derivative thereof.

A variety of decalcifying formulations are provided for reducing the calcification of a tissue in a root canal of a patient in need thereof. The decalcifying formulation can include an effective amount of Cysteamine or a derivative thereof to reduce the calcification of the tissue in the root canal of the patient. The effective amount can be effective to unblock the root canal to a greater extent or as compared to the otherwise same formulation except without the Cysteamine or Cysteamine derivative. The effective amount can be effective to unblock the root canal to the same extent but in less time as compared to the otherwise same formulation except without the Cysteamine or Cysteamine derivative. The effective amount can be effective to unblock the root canal to a greater extent (or to the same extent but in less time) as compared to the otherwise same formulation except with the Cysteamine or Cysteamine derivative replaced with NaOCl in an amount ranging from about 6.15% to about 8.25% (v/v) based upon the volume of the formulation. The effective amount can be effective to reduce an amount of calcified tissue (%) in the root canal to about 10% or less when the root canal is exposed to the formulation for a period of time of at least 48 hours to about 240 hours. In some cases, the amount of calcified tissue (%) prior to the treatment is at least 50% or more.

The Cysteamine or Cysteamine derivative can be present in any amount that is effective for the application and patient. In some aspects, the Cysteamine or Cysteamine derivative is present in an amount from about 6 mg/ml to 16 mg/ml, about 8 mg/ml to 16 mg/ml, about 8 mg/ml to 14 mg/ml, about 8 mg/ml to 12 mg/ml, about 8 mg/ml to 10 mg/ml, or about 6 mg/ml to 10 mg/ml.

In some aspects, the effective amount is effective to reduce the bacterial proliferation in the root canal wall of the patient as compared to a bacterial proliferation of the otherwise same root canal wall when treated with the otherwise same irrigant formulation except with the Cysteamine or Cysteamine derivative replaced with NaOCl in an amount ranging from about 6.15% to about 8.25% (v/v) based upon the volume of the formulation. In some aspects, the bacterial proliferation is selected from the group consisting of Enterococcus faecalis, Fusobacterium nucleatum, and a combination thereof. In some aspects, the effective amount is effective to produce a percent reduction in Enterococcus faecalis in the canal wall of the patient of about 50%, about 55%, about 60%, about 65%, or more when administered to the patient. In some aspects, the effective amount is effective to produce a percent reduction in Fusobacterium nucleatum in the canal wall of the patient of about 50%, about 55%, about 60%, about 65%, or more when administered to the patient.

In some aspects, the irrigant contains Cysteamine, having the chemical formula H₂NCH₂CH₂SH. In some aspects, the formulations contain a Cysteamine derivative. In some aspects, the Cysteamine or Cysteamine derivative can be described by the general formula

H₂N-A-SH

where A is a substituted or an unsubstituted C₁-C₇ alkyl, a substituted or an unsubstituted C₂-C₇ alkenyl, a substituted or an unsubstituted C₂-C₇ alkynyl, a substituted or an unsubstituted C₃-C₇ cycloalkyl, a substituted or an unsubstituted C₃-C₇ cycloalkenyl, or a substituted or an unsubstituted C₃-C₇ alkynyl. In some aspects, A is a substituted or unsubstituted alkyl group having 2, 3, 4, 5, or 6 carbon atoms in the backbone.

In some aspects, the irrigant formulation includes an additional decalcifying agent. There are two categories of decalcifying agents namely, chelating agents and acids. The acids are further divided into weak (picric, acetic and formic acid) and strong acids (nitric and hydrochloric acid). The acids make up a solution of calcium ions while the chelating agents take up the calcium ions. Some common additional decalcifying agents used in endodontic applications can include ethylene diamine tetra acetic acid (EDTA), citric acid, hydroxy ethylidene-bisphosphonate (HEBP).

In some aspects, the formulations include a surfactant. Surfactants can aide in the removal of larger particulate in the smear layer. “Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

The Cysteamine or Cysteamine derivative can be formulated for topical administration. Suitable dosage forms for topical administration include creams, ointments, salves, sprays, gels, lotions, emulsions, and liquids. The compositions can contain one or more chemical penetration enhancers, membrane permeability agents, membrane transport agents, emollients, surfactants, stabilizers, and combination thereof.

In some aspects, the Cysteamine or Cysteamine derivative can be administered as a liquid formulation, such as a solution or suspension, a semi-solid formulation, such as a lotion or ointment, or a solid formulation. In some aspects, the Cysteamine or Cysteamine derivative is formulated as liquid, including solutions and suspensions, or as a semi-solid formulation, such as ointment or lotion for topical application.

The formulation may contain one or more excipients, such as emollients, surfactants, emulsifiers, penetration enhancers, and the like.

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one aspect, the emollients are ethylhexylstearate and ethylhexyl palmitate.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one aspect, the emulsifier is glycerol stearate.

Suitable classes of penetration enhancers are known in the art and include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols). Examples of these classes are known in the art.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

An “emulsion” is a composition containing a mixture of non-miscible components homogenously blended together. In particular aspects, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one aspect, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one aspect, the formulation is in the form of a cream having a viscosity of greater than 1000 centistokes, typically in the range of 20,000-50,000 centistokes. Creams are often time preferred over ointments as they are generally easier to spread and easier to remove.

The difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of the Cysteamine or Cysteamine derivative in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C₁₂-C₁₅ alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

Irrigation Methods

In various aspects, methods of irrigating a patient in need thereof are provided. The methods can include applying a formulation describe above. The methods can provide an effective amount of the Cysteamine or the Cysteamine derivative to the patient in need thereof. The methods can include removal of the smear layer of the root canal wall. The methods can provide for effective inhibition of bacterial proliferation in the root canal wall of the patient or subject.

Decalcifying Methods

In various aspects, methods are provided for decalcifying a calcified tissue in a root canal of a patient in need thereof. The methods can include applying a formulation describe above. The methods can provide an effective amount of the Cysteamine or the Cysteamine derivative to the patient in need thereof. The methods can be performed as part of a root canal procedure, with or without the application of a dental crown.

### EXAMPLES

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of aspects of the present disclosure.

### Example 1

### Bacterial Removal from Root Canal Systems Using Cysteamine as Compared to NaOCl

Methods

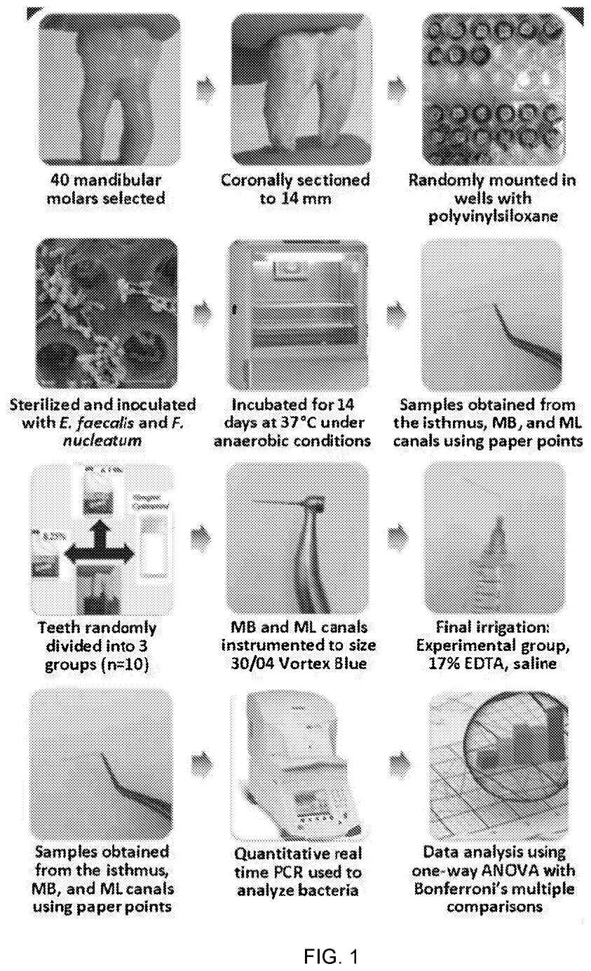

Forty human mandibular molars were accessed, sterilized, inoculated with Enterococcus faecalis (OG1RF) and Fusobacterium nucleatum (ATCC10953), and incubated for 14 days under anaerobic conditions. Teeth were divided into 3 groups (n=10): 1) 6.15% NaOCl, 2) 8.25% NaOCl, 3) Cysteamine. Controls (n=5): negative without inoculation and positive without instrumentation/irrigation. Mesiobuccal and mesiolingual canals were instrumented using Vortex Blue to 30/.04 and the corresponding irrigant, followed by neutralization with EDTA and saline. Samples were collected pre- and post-instrumentation/irrigation. Total DNA was extracted and quantitative real-time PCR was performed. Data were analyzed using one-way ANOVA with Bonferroni's multiple comparisons (p<0.05). See FIG. 1 for a schematic representation of the methods.

Results

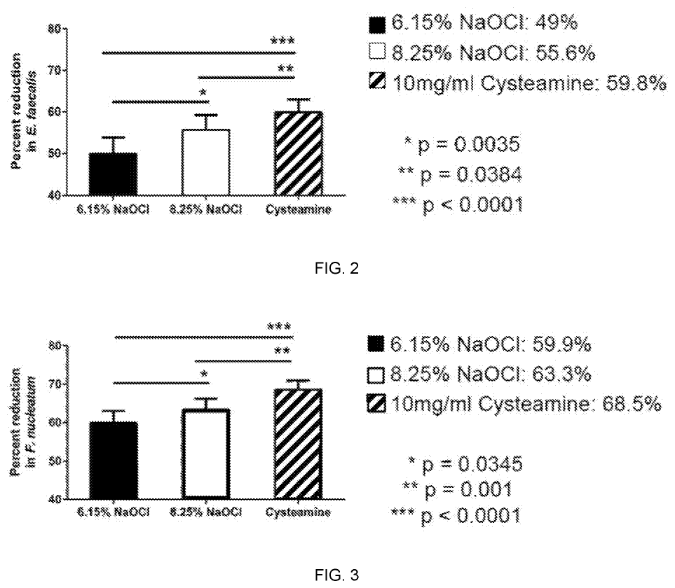

The results for the percent reduction in Enterococcus faecalis are presented in FIG. 2. Results for the percent reduction in Fusobacterium nucleatum are presented in FIG. 3. Results showed that 6.15% NaOCl reduced Enterococcus faecalis and Fusobacterium nucleatum by 49% and 59.9% respectively; 8.25% NaOCl by 54% and 63.4%; and Cysteamine by 60% and 68.5%. We concluded that Cysteamine promotes more Enterococcus faecalis and Fusobacterium nucleatum reduction than 6.15% or 8.25% NaOCl.

### PROPHETIC EXAMPLES

Now having described examples of the present disclosure, in general, the following Prophetic Examples describe some additional aspects which should not be confused with the Examples. While the examples are described prophetically, this is not intended to limit aspects of the present disclosure. On the contrary, the intent is to cover the prophetic examples as if the results were fully presented herein. The methods and procedures are described in sufficient detail to be performed reproducibly by those skilled in the art as if the results were fully presented herein.

### Prophetic Example 1

### In Vivo Study of Canal Cleanliness and Antimicrobial Efficacy with Cysteamine

The aim of this study is to identify the bacteria that remains colonizing the root canal system following different irrigation techniques using 454-sequencing and qPCR.

Techniques:

Pyrosequencing: Massive Parallel Pyrosequencing will be utilized to increase the efficiency of DNA sequencing of bacteria which colonize the dental pulp. This allows for direct interrogation of the microbial genomes by comparison of amplified orthologous gene sequences without requiring the cultivation of microbes. Specifically, bacterial 16s tag-encoded FLX-titanium amplicon pyrosequencing (bTEFAP) will be performed using the 27F and 338R universal primers, which target the V1-V2 hyper-variable regions of 16S rRNA genes (Integrated DNA Technologies, San Diego, Calif.). The 338R primer includes a unique 12-base pair sequence tag, known as a barcode, for each sample. Thus, each DNA sample undergoing pyrosequencing will acquire a unique barcode, which allows amplified bacterial sequences to be traced back to a specific clinical sample. Following PCR amplification of the V1-V2 hyper-variable regions and barcoding of each product, PCR products will be submitted to the Bioinformatics and Genomics Center at the University of Florida, for pyrosequencing on the Roche 454 GS FLX instrument. Sequencing data will be analyzed by using the Quantitative Insights into Microbial Ecology (QIIME) software whereby bacterial identification, will be performed at the finest level of taxonomic resolution possible given the sequence reliability and current state of databases. All qualitative data generated by 454-pyrosequencing will be confirmed and quantified using qPCR.

Real-time PCR: qPCR will be used to determination the number of copies or relative expression of genes of interest. Here, total genomic bacterial DNA will be extracted from paper points using Qiagen DNA extraction kit, according to the manufacturer's instructions. After which bacterial specific qPCR primers will be used to determine the relative expression of the bacterial species normalized to total 16S. Normalized expression=[[corrected Cₜ value of sample of interest]/[corrected Cₜ value of non-traumatic sample]−1]. Corrected Cₜ value=[raw Cₜ value]×[16S corrective ratio]. 16S corrective ratio=[lowest 16S Cₜ within sample set]/[16S copy number for cell of interest].

Statistical analyses: Following PCR amplification of the V1−V2 hyper-variable regions and barcoding of each product, PCR products will be submitted to the Bioinformatics and Genomics Center at the University of Florida, for pyrosequencing on the Roche 454 GS FLX instrument. Sequencing data will be analyzed by using the Quantitative Insights into Microbial Ecology (QIIME) software whereby bacterial identification, will be performed at the finest level of taxonomic resolution possible given the sequence reliability and current state of databases. All qualitative data generated by 454-pyrosequencing will be confirmed and quantified using qPCR.

Material and Methods:

Eighty extracted mandibular molars will be used for this study. The teeth will be supplied by the company and each will be placed into a 2 ml aliquot of modified Amies transport medium. Samples will be shipped on ice by overnight carrier. This transport medium maintains anaerobosis but does not promote outgrowth of the bacteria present. Once received, teeth will be stored refrigerated until processed.

Pre-Microbial Sample:

Teeth will be randomized into four different groups, accessed with high speed bur, and a microbial pre-sample will be taken with a fine paper point to the length of the root. Additional groups can be included to assess additional formulations described herein.

Irrigation:

Working length will be determined by placing a size 10 file into the canal until visualized at the apex, and subtracting 1.00 mm from this measurement. Canals will be coronally opened with a gates-gliddden bur sizes 1 and 2 to enable full penetration of the irrigation solutions to the apical third of the canal system. The specimens will be mounted in a alginate impression, so the apex were blocked in a method to simulate the occlusion of the tissue, PDL, or lesion pressure and not allow free flow of irrigants out the apex. The irrigation technique for all samples will be 2 ml for 60 seconds using a 28 gauge Pro Rinse needle (Dentsply Tulsa Dental Specialties, Tulsa Okla.) with a final sonic activation of the respective irrigation solution using endo activator for 60 seconds.

Since an objective is to determine the effectiveness of the irrigation solution in dentistry the canal will not receive mechanical instrumentation with any type of files.

Irrigation Groups:

Group I: Following access and microbial pre-sampling teeth will be irrigated with 2 ml of 8.25% NaOCl, 2 ml of EDTA, and 2 ml of 2% CHX (n=10). The final sonication will be with 2% CHX.

Group II : Following access and microbial pre-sampling teeth will be irrigated with 2 ml of 8.25% NaO0Cl followed by Qmix for 60 seconds and sonicated with Qmix (n=10).

Group III : Following access and microbial pre-sampling, teeth will be irrigated with 2 ml of Cysteamine for 60 seconds using Endoactivator agitation with Cysteamine (n=10).

Group IV: Following access and pre-sampling, teeth will be irrigated with 2 ml of saline (n=10).

Additional groups can be included to assess the efficacy of any formulation described herein. Following access and microbial pre-sampling, teeth can then be irrigated with 2 ml of any formulation described herein for 60 seconds using Endoactivator agitation with (n=10).

Antimicrobial Analysis:

Prior to instrumentation, a paper point will be placed in both canals (MB and ML) and isthmus for 10 seconds for bacterial sample collection and quantification. The three paper points will be then transferred into sterile eppendorf tubes containing 1 mL of sterile sodium-magnesium buffer (pH 7.4) after which bacterial DNA will be extracted and the resulting samples will be analyzed by 454 Pyrosequencing. All findings will be validated and quantified by real-time PCR (qPCR).

Following instrumentation and irrigation, a post treatment sample will be taken and analyzed following the same protocol. Therefore, we will qualitatively and quantitatively evaluate the colonization of bacteria in the root canal system prior and after instrumentation using 454-sequencing and qPCR.

### Prophetic Example 2

### Reduction of a Polymicrobial Biofilm

The aim of this study is to evaluate the reduction of a polymicrobial biofilm composed of Enterococcus Faecalis and Fusobacterium Nucleatum.

Materials and Methods:

Ten teeth in each group will be accessed with Gates Glidden, sterilized, and inoculated with a polymicrobial biofilm. The polymicrobial biofim composed of E. faecalis (ATCC 29212) and F. nucleatum (ATCC 25581) will be prepared for inoculation into the sterilized canals of each tooth, except for the negative controls. The bacterial suspensions will be injected into each root canal using a sterile syringe and a 30 gauge irrigation needle (Max-i-Probe—Dentsply Rinn, Elgin, Ill.). After inoculation, all teeth will be incubated at 37° C. in a CO₂ chamber to allow growth biofilm for 10 days.

Following biofilm formation, teeth will be instrumented following the experimental group protocol from Prophetic Example 1, with samples collected with paper points pre and post-instrumentation/irrigation. Following extraction of total DNA, quantitative real-time PCR will be used to analyze presence of E. faecalis and F. nucleatum. Data will be normalized and analyzed using one-way Anova with Bonferroni's correction for multiple comparisons (p<0.05).

### Prophetic Example 3

### Effect on the Removal of Calcified Dental Tissue

Elderly patients present to the clinician more challenges during endodontic treatment. Despite the currently increased number of an older population that has issues with staying comfortably on a dental chair for longer periods of time, they also bring a complex systemic involvement which often leads to an increase in calcified tissues within the canal space. When the anatomy of the canal space is calcified, and cleanliness of the entire length of the tooth is unachievable, treatment outcome is substantially reduced. When infection is present and the patients have undergone chemotherapy treatment and/or radiation treatment, elimination of infection is of paramount importance, since under these circumstances the teeth cannot be extracted due to a predisposition for osteonecrosis of the jaw.

Due to the calcific barrier, conventional method for glide path creation involving the use of manual hand instrumentation with stainless steel(SS) K-files up to a size #10 or 15 to the entire length of the canal is impossible. Alternatively, newer approaches suggest limiting hand instrumentation to initial canal negotiation with a maximum size in conjunction with a large amounts of canal irrigation with EDTA, a chelating agent, but still not proven to be successful. The aim of this study is to measure the working time, occurrence of complete canal decalcification when three different irrigation solutions are left in the canal system for a week with the purpose of disrupting this calcific barrier and promoting complete cleanliness of the canal system.

Material and Methods: Thirty-seven mandibular molars that following access cannot be instrumented to a file size 8 to complete working length of the canal will be included in this study. The canals which met inclusion criteria were randomly divided into 4-groups with 16 canals each.

Irrigation Groups:

Group I: Following access teeth will be irrigated with 2 ml of 8.25% NaOCl, 2 ml of EDTA, and 2 ml of 2% CHX (n=10). The final sonication will be with 2% CHX and the last solution left in the canal for one week.

Group II : Following access teeth will be irrigated with 2 ml of 8.25% NaOCl followed by Qmix for 60 seconds and sonicated with Qmix (n=10), and solution left in the canal space for a week.

Group III : Following access, teeth will be irrigated with 2 ml of Cysteamine for 60 seconds using Endoactivator agitation with Cysteamine (n=10), and Cysteamine left in the canal for a week.

Group IV: Following access and pre-sampling, teeth will be irrigated with 2 ml of saline (n=10), and left in the canal for a week, serving as a control.

Additional groups can be included to assess the efficacy of any formulation described herein. Following access, teeth can then be irrigated with 2 ml of any formulation described herein for 60 seconds using Endoactivator agitation (n=10), and the formulation can be left in the canal for a week.

Decalcification Specimen Analysis:

The understanding of root canal anatomy is paramount for a successful root canal therapy outcome. The time and ability to unblock each canal to full length with a K file size #s 8 and 10 will be measured and data individually statistically analyzed.

Then, also following the irrigation assessment, the samples will be prepared for SEM analysis.

Data Analysis:

All the sections will be analyzed following a grade scale:

1. No remaining calcified debris left

2. Minimum debris left (1-10%)

3. Medium amount of debris (10-50%)

4. Significant amount of debris left (50%)

The results will be evaluated by three blinded investigators and statistically analyzed using a Kruskall-Wallis Test and a Dunn's Multiple test.

Imaging at the nanoscale level will improve our understanding of the canal structures and may reveal different properties and characteristics from our current understanding. With the use of the SEM we can examine the detailed topography within the root canal system, allowing us to observe the canal anatomical variations related to blockage in greater detail.

Samples of each group will be sectioned longitudinally. Teeth will be decoronated at the cervical line using an Isomet Buehler slow speed saw. The roots will be splited with a hammer and chisel into two halves. Samples will be coded so that the test and control teeth cannot be identified during the evaluation process and examined under microscope.

It should be emphasized that the above-described aspects of the present disclosure are merely possible examples of implementations, and are set forth only for a clear understanding of the principles of the disclosure. Many variations and modifications may be made to the above-described aspects of the disclosure without departing substantially from the spirit and principles of the disclosure. All such modifications and variations are intended to be included herein within the scope of this disclosure.

The present disclosure will be better understood upon review of the following features, which should not be confused with the claims.

Feature 1. An endodontic formulation suitable for administration to a patient in need thereof, the endodontic formulation comprising Cysteamine or a derivative thereof.

Feature 2. The endodontic formulation according to Feature 1, wherein the formulation is an irrigant formulation for reducing a bacterial proliferation in a root canal wall of the patient.

Feature 3. The endodontic formulation according to Feature 1, wherein the formulation is a decalcifying formulation for reducing a calcification of a tissue in a root canal of the patient.

Feature 4. An irrigant formulation for reducing a bacterial proliferation in a root canal wall of a patient in need thereof, the irrigant formulation comprising: an effective amount of Cysteamine or a derivative thereof to reduce the bacterial proliferation in the root canal wall of the patient as compared to a bacterial proliferation of the otherwise same root canal wall when treated with the otherwise same irrigant formulation except without the Cysteamine or derivative thereof.

Feature 5. A decalcifying formulation for reducing a calcification of a tissue in a root canal of a patient in need thereof, the decalcifying formulation comprising: an effective amount of Cysteamine or a derivative thereof to reduce the calcification of the tissue in the root canal of the patient.

Feature 6. The formulation according to any one of Features 1-5, wherein the Cysteamine or Cysteamine derivative is present in an amount from about 6 mg/ml to about 14 mg/ml, about 8 mg/ml to about 12 mg/ml, or about 9 mg/ml to about 11 mg/ml.

Feature 7. The formulation according to any one of Features 1-6, wherein the formulation comprises a Cysteamine derivative having a structure according to the following formula:

H₂N-A-SH

where A is selected from the group consisting of substituted and unsubstituted C₁-C₇ alkyl, substituted and unsubstituted C₂-C₇ alkenyl, substituted and unsubstituted C₂-C₇ alkynyl, substituted and unsubstituted C₃-C₇ cycloalkyl, substituted and unsubstituted C₃-C₇ cycloalkenyl, and substituted and unsubstituted C₃-C₇ cycloalkynyl.

Feature 8. The formulation according to Feature 7, wherein A is selected from the group consisting of substituted and unsubstituted C₂-C₅ alkyl, substituted and unsubstituted C₁-C₅ alkyl, substituted and unsubstituted C₂-C₇ alkyl, substituted and unsubstituted C₃-C₇ alkyl, and substituted and unsubstituted C₂-C₆ alkyl.

Feature 9. The formulation according to any one of Features 1-8, further comprising an additional decalcifying agent.

Feature 10. The formulation according to Feature 9, wherein the additional decalcifying agent is selected from the group consisting of ethylene diamine tetra acetic acid (EDTA), citric acid, hydroxy ethylidene-bisphosphonate (HEBP),

Feature 11. The formulation according to any one of Features 1-10, further comprising a surfactant.

Feature 12. The formulation according to any one of Features 1-11, wherein the effective amount is effective to reduce the bacterial proliferation in the root canal wall of the patient as compared to a bacterial proliferation of the otherwise same root canal wall when treated with the otherwise same irrigant formulation except with the Cysteamine or Cysteamine derivative replaced with NaOCl in an amount ranging from about 6.15% to about 8.25% (v/v) based upon the volume of the formulation.

Feature 13. The formulation according to any one of Features 1-12, wherein the bacterial proliferation is selected from the group consisting of Enterococcus faecalis, Fusobacterium nucleatum, and a combination thereof.

Feature 14. The formulation according to any one of Features 1-13, wherein the effective amount is effective to produce a percent reduction in Enterococcus faecalis in the canal wall of the patient of about 40%, about 50%, about 60%, about 65%, about 70% or more when administered to the patient.

Feature 15. The formulation according to any one of Features 1-14, wherein the effective amount is effective to produce a percent reduction in Fusobacterium nucleatum in the canal wall of the patient of about 40%, about 50%, about 60%, about 65%, about 70% or more when administered to the patient.

Feature 16. The formulation according to any one of Features 1-15, wherein the effective amount is effective to unblock the root canal to a greater extent or to the same extent but in less time as compared to the otherwise same formulation except without the Cysteamine or Cysteamine derivative.

Feature 17. The formulation according to any one of Features 1-16, wherein the effective amount is effective to unblock the root canal to a greater extent or to the same extent but in less time as compared to the otherwise same formulation except with the Cysteamine or Cysteamine derivative replaced with NaOCl in an amount ranging from about 6.15% to about 8.25% (v/v) based upon the volume of the formulation.

Feature 18. The formulation according to any one of Features 1-17, wherein the effective amount is effective to reduce an amount of calcified tissue (%) in the root canal to about 20%, about 15%, about 10% or less when the root canal is exposed to the formulation for a period of time of at least 24 hours to about 240 hours, at least 48 hours to about 240 hours at least 72 hours to about 240 hours.

Feature 19. A method of irrigating a root canal wall of a patient in need thereof, the method comprising administering an effective amount of a formulation according to any one of Features 1-18 to reduce a bacterial proliferation in the root canal wall of the patient.

Feature 20. A method of irrigating a root canal wall of a patient in need thereof, the method comprising administering an effective amount of Cysteamine or a derivative thereof to reduce the bacterial proliferation in the root canal wall of the patient as compared to a bacterial proliferation of the otherwise same root canal wall when treated in the otherwise same manner except without administering the Cysteamine or derivative thereof.

Feature 21. A method of reducing a calcification of a tissue in a root canal of a patient in need thereof, the method comprising administering an effective amount of a formulation according to any one of Features 1-18 to the root canal for a period of time sufficient to reduce the calcification of the tissue in the patient.

Feature 22. A method of reducing a calcification of a tissue in a root canal of a patient in need thereof, the method comprising administering an effective amount of Cysteamine or a derivative thereof to the root canal for a period of time sufficient to reduce the calcification of the tissue in the patient.

Feature 23. The method according to any one of Features 21-22, wherein the period of time is about 24 hours to about 240 hours, about 48 hours to about 240 hours, or about 72 hours to about 240 hours.

Feature 24. The method according to any one of Features 19-23, wherein the Cysteamine or Cysteamine derivative has a structure according to the following formula:

H₂N-A-SH

where A is selected from the group consisting of substituted and unsubstituted C₁-C₇ alkyl, substituted and unsubstituted C₂-C₇ alkenyl, substituted and unsubstituted C2-C7 alkynyl, substituted and unsubstituted C₃-C₇ cycloalkyl, substituted and unsubstituted C₃-C₇ cycloalkenyl, and substituted and unsubstituted C₃-C₇ alkynyl.

Feature 25. The method according to Feature 24, wherein A is selected from the group consisting of substituted and unsubstituted C₂-C₅ alkyl, substituted and unsubstituted C₁-C₅ alkyl, substituted and unsubstituted C₂-C₇ alkyl, substituted and unsubstituted C₃-C₇ alkyl, and substituted and unsubstituted C₂-C₆ alkyl.

Feature 26. The method according to any one of Features 19-25, wherein the effective amount is effective to reduce the bacterial proliferation in the root canal wall of the patient as compared to a bacterial proliferation of the otherwise same root canal wall when treated with the otherwise same method except with the Cysteamine or Cysteamine derivative replaced with NaOCl in an amount ranging from about 6.15% to about 8.25% (v/v) based upon the volume of the formulation.

Feature 27. The method according to any one of Features 19-26, wherein the bacterial proliferation is selected from the group consisting of Enterococcus faecalis, Fusobacterium nucleatum, and a combination thereof.

Feature 28. The method according to any one of Features 19-27, wherein the effective amount is effective to produce a percent reduction in Enterococcus faecalis in the canal wall of the patient of about 40%, about 50%, about 60%, about 65%, about 70% or more when administered to the patient.

Feature 29. The method according to any one of Features 19-28, wherein the effective amount is effective to produce a percent reduction in Fusobacterium nucleatum in the canal wall of the patient of about 40%, about 50%, about 60%, about 65%, about 70% or more when administered to the patient.

1. An endodontic formulation suitable for administration to a patient in need thereof, the endodontic formulation comprising an effective amount of Cysteamine or a derivative thereof.

2. The endodontic formulation according to claim 1, wherein the formulation is an irrigant formulation for reducing a bacterial proliferation in a root canal wall of the patient:
wherein the reduction of the bacterial proliferation is compared to a bacterial proliferation of the otherwise same root canal wall when treated in the otherwise same manner except without administering the Cysteamine or derivative thereof; or
wherein the reduction of the bacterial proliferation is compared to a bacterial proliferation of the otherwise same root canal wall when treated with the otherwise same irrigant formulation except with the Cysteamine or Cysteamine derivative replaced with NaOCl in an amount ranging from about 6.15% to about 8.25% (v/v) based upon the volume of the formulation.

3. The endodontic formulation according to claim 1, wherein the formulation is a decalcifying formulation for reducing a calcification of a tissue in a root canal of the patient:
wherein the effective amount is effective to unblock the root canal to a greater extent or to the same extent but in less time as compared to the otherwise same formulation except without the Cysteamine or Cysteamine derivative; or
wherein the effective amount is effective to unblock the root canal to a greater extent or to the same extent but in less time as compared to the otherwise same formulation except with the Cysteamine or Cysteamine derivative replaced with NaOCl in an amount ranging from about 6.15% to about 8.25% (v/v) based upon the volume of the formulation.

4-5. (canceled)

6. The formulation according to claim 1, wherein the Cysteamine or Cysteamine derivative is present in an amount from about 8 mg/ml to about 12 mg/ml.

7. The formulation according to according to claim 1, wherein the formulation comprises a Cysteamine derivative having a structure according to the following formula:
H₂N-A-SH
where A is selected from the group consisting of substituted and unsubstituted C₁-C₇ alkyl, substituted and unsubstituted C₂-C₇ alkenyl, substituted and unsubstituted C₂-C₇ alkynyl, substituted and unsubstituted C₃-C₇ cycloalkyl, substituted and unsubstituted C₃-C₇ cycloalkenyl, and substituted and unsubstituted C₃-C₇ alkynyl.

8. The formulation according to claim 7, wherein A is selected from the group consisting of substituted and unsubstituted C₂-C₅ alkyl.

9. The formulation according to claim 1, further comprising an additional decalcifying agent.

10. The formulation according to claim 9, wherein the additional decalcifying agent is selected from the group consisting of ethylene diamine tetra acetic acid (EDTA), citric acid, hydroxy ethylidene-bisphosphonate (HEBP).

11-12. (canceled)

13. The formulation according to claim 2, wherein the bacterial proliferation is selected from the group consisting of Enterococcus faecalis, Fusobacterium nucleatum, and a combination thereof.

14. The formulation according to claim 13, wherein the effective amount is effective to produce a percent reduction in Enterococcus faecalis in the canal wall of the patient of about 60% or more when administered to the patient.

15. The formulation according to claim 13, wherein the effective amount is effective to produce a percent reduction in Fusobacterium nucleatum in the canal wall of the patient of about 65% or more when administered to the patient.

16-17. (canceled)

18. The formulation according to claim 3, wherein the effective amount is effective to reduce an amount of calcified tissue (%) in the root canal to about 10% or less when the root canal is exposed to the formulation for a period of time of at least 48 hours to about 240 hours.

19. (canceled)

20. A method of irrigating a root canal wall of a patient in need thereof, the method comprising administering an effective amount of Cysteamine or a derivative thereof to reduce the bacterial proliferation in the root canal wall of the patient as compared to a bacterial proliferation of the otherwise same root canal wall when treated in the otherwise same manner except without administering the Cysteamine or derivative thereof.

21. (canceled)

22. A method of reducing a calcification of a tissue in a root canal of a patient in need thereof, the method comprising administering an effective amount of Cysteamine or a derivative thereof to the root canal for a period of time sufficient to reduce the calcification of the tissue in the patient.

23. The method according to claim 22, wherein the period of time is about 48 hours to about 240 hours.

24. The method according to claim 20, wherein the Cysteamine or Cysteamine derivative has a structure according to the following formula:
H₂N-A-SH
where A is selected from the group consisting of substituted and unsubstituted C₁-C₇ alkyl, substituted and unsubstituted C₂-C₇ alkenyl, substituted and unsubstituted C2-C7 alkynyl, substituted and unsubstituted C₃-C₇ cycloalkyl, substituted and unsubstituted C₃-C₇ cycloalkenyl, and substituted and unsubstituted C₃-C₇alkynyl.

25. (canceled)

26. The method according to claim 20, wherein the effective amount is effective to:
reduce the bacterial proliferation in the root canal wall of the patient as compared to a bacterial proliferation of the otherwise same root canal wall when treated with the otherwise same method except with the Cysteamine or Cysteamine derivative replaced with NaOCl in an amount ranging from about 6.15% to about 8.25% (v/v) based upon the volume of the formulation; or
reduce the bacterial proliferation in the root canal wall of the patient as compared to a bacterial proliferation of the otherwise same root canal wall when treated with the otherwise same method except without administering the Cysteamine or derivative thereof.

27. The method according to claim 20, wherein the bacterial proliferation is selected from the group consisting of Enterococcus faecalis, Fusobacterium nucleatum, and a combination thereof.

28. The method according to claim 27, wherein the effective amount is effective to produce a percent reduction in Enterococcus faecalis of the bacterial proliferation in the canal wall of the patient of about 60% or more when administered to the patient.

29. (canceled)

30. The method according to claim 22, wherein the Cysteamine or Cysteamine derivative has a structure according to the following formula:
H₂N-A-SH
where A is selected from the group consisting of substituted and unsubstituted C₁-C₇ alkyl, substituted and unsubstituted C₂-C₇ alkenyl, substituted and unsubstituted C2-C7 alkynyl, substituted and unsubstituted C₃-C₇ cycloalkyl, substituted and unsubstituted C₃-C₇ cycloalkenyl, and substituted and unsubstituted C₃-C₇ alkynyl.
